(19) United States
(12) Patent Application Publication
(10) Pub. No.: US 2025/0345106 A1
(43) Pub. Date: Nov. 13, 2025

(54) BENDABLE ORTHOPEDIC FASTENERS

(71) Applicant: SG, LLC, Highland Beach, FL (US)

(72) Inventors: Paul Glazer, Highland Beach, FL (US); Michael J. Milella, JR., Imperial Beach, CA (US)

(73) Assignee: SG, LLC, Highland Beach, FL (US)

(21) Appl. No.: 19/274,184

(22) Filed: Jul. 18, 2025

Related U.S. Application Data
(63) Continuation-in-part of application No. 18/126,201, filed on Mar. 24, 2023, now Pat. No. 12,364,525.

(51) Int. Cl. A61B 17/84; A61B 17/00; A61B 17/68

(52) U.S. Cl. CPC A61B 17/84; A61B 17/686; A61B 2017/00982

(57) ABSTRACT

In accordance with at least one aspect of this disclosure, a flexible fastener includes, a shaft having a proximal end and a distal end spaced apart along a longitudinal axis and a land surface winding helically around the shaft. An interior pocket extends in an axial direction inside the shaft, radially inward from the land surface. A flexure opening extends through the shaft in the radial direction from the land surface to an inward facing surface of the interior pocket. The flexure opening extends helically about the longitudinal axis to provide for flexure of the shaft, of the land surface, and of the external thread. A torque driver is seated in the interior pocket, the toque driver having a torque face configured to abut a torque face of the interior pocket to develop toque along the shaft when a driving torque is applied to the proximal end of the shaft.

### CROSS-REFERENCE TO RELATED APPLICATIONS

This application is a continuation-in-part of U.S. patent application Ser. No. 18/126,201, filed Mar. 23, 2023, the entire content of which is incorporated herein by reference.

### BACKGROUND

### 1. Field

The present disclosure relates to fasteners, and more particularly to flexible fasteners such as for use in orthopedic applications.

### 2. Description of Related Art

A typical screw includes a shaft extending from a head. A helical thread winds around the shaft so that torque applied to the head rotates the threads to drive the screw into two or more substrates to join the substrates together. In some cases, one or more of the substrates may be prepared with a female thread for engaging the male thread of the screw. Other screws are self-tapping and do not require pre-threaded bores in the substrates being joined.

There is a variety of orthopedic application for screws, i.e. bone screws. Bone screws can be used to join to pieces of a bone together, e.g. such as in treatment of trauma. In other procedures, bone screws can be used to anchor an implant to bone, such as in an artificial hip replacement or a spinal implant.

Some bendable fasteners such as screws have allowed for a bone screw to flex as it is being driven so that the tip of the screw ultimately points a different direction from the driver driving the head. This can be beneficial, for example where minimally invasive surgical site access limits a surgeon's mobility with the driver. Example applications of a bendable screws and other fasteners for various implants are described in U.S. Pat. No. 9,597,199 to Glazer.

There has been a tradeoff in designing bendable screws using conventional methods. The more flexible the fastener, the more likely it is to break while being driven. The stronger the fastener is for transmitting torque and avoiding breakage while being driven, the less flexible it tends to be.

The conventional techniques have been considered satisfactory for their intended purpose. However, there is an ever-present need for improved systems and methods for bendable fasteners such as for use in orthopedic applications. This disclosure provides a solution for this need.

### SUMMARY

In accordance with at least one aspect of this disclosure, a flexible fastener includes, a shaft having a proximal end and a distal end spaced apart along a longitudinal axis and a land surface winding helically around the shaft. An interior pocket extends in an axial direction inside the shaft, radially inward from the land surface. A flexure opening extends through the shaft in the radial direction from the land surface to an inward facing surface of the interior pocket. The flexure opening extends helically about the longitudinal axis to provide for flexure of the shaft, of the land surface, and of the external thread. A torque driver is seated in the interior pocket, the toque driver having a torque face configured to abut a torque face of the interior pocket to develop toque along the shaft when a driving torque is applied to the proximal end of the shaft.

In embodiments, the torque driver can be free floating (e.g., not integral with or affixed to) within the interior pocket when the shaft is in a relaxed state where no torque is applied to the shaft. In embodiments, the torque driver can include a proximal portion and a distal portion with a narrowing neck connecting the distal portion to the proximal portion. In certain embodiments, the torque face of the torque driver can be a first torque face of the proximal portion of the torque driver and the proximal portion of the torque driver can include at least one additional torque face. In embodiments, the distal portion of the torque driver can include a plurality of torque faces.

In embodiments, the interior pocket can include a proximal portion housing the proximal portion of the torque driver. The interior pocket can also include a distal portion housing the distal portion of the torque driver. In embodiments, the interior pocket can define a narrowing neck connecting between the proximal and distal portions of the interior pocket through which the narrowing neck of the torque driver passes.

In embodiments, each of the torque faces of the distal portion of the torque driver can face a corresponding torque face of the distal portion of the interior pocket for abutment. In embodiments, each of the torque faces of the proximal portion of the torque driver can face a corresponding torque face of the proximal portion of the interior pocket for abutment.

In embodiments, a clearance gap can be defined between the toque driver and the interior pocket so the toque driver can move within the pocket as long as the shaft is in the relaxed state. The clearance gap, torque faces of the torque driver, and the torque faces of the interior pocket can be configured so the clearance gap is too small to allow the rotation of the torque driver about the longitudinal axis beyond a point where the torque faces of the interior pocket and of the torque driver come into abutment.

The distal and proximal portions of the torque driver can define a square axial cross-section with a diagonal too large to rotate within the interior pocket beyond abutment of the torque faces. In certain embodiments, abutment (e.g., as used here) may not necessarily require full planar contact or engagement. In embodiments, the proximal and distal portions of the torque driver can have a cul-de-sac cross-sectional shapes in a plane of the diagonal. In certain embodiments, the proximal and distal portions of the torque driver can be aligned along the same diagonal. In certain embodiments the proximal and distal portions of the torque driver can be clocked about the longitudinal axis relative to one another.

In certain embodiments, the narrowing neck of the torque driver can define a circular axial cross-section. In certain embodiments, the flexure opening can be too small to admit the torque driver therethrough so the torque driver is captured in the interior pocket. In embodiments, a proximal end of the flexure opening can be axially proximate a proximal end of the interior pocket with respect to the longitudinal axis. In embodiments, the flexure opening can wind around the interior pocket to a distal end of the flexure opening that is proximal to a distal end of the interior pocket. In certain embodiments, the flexure opening can bend beyond 360° around the interior pocket circumferentially relative to the longitudinal axis. In embodiments, the distal end of the flexure opening can define a distal stress-reducing cul-de-sac shape, and the proximal end of the flexure opening can define a proximal stress-reducing cul-de-sac shape.

In embodiments, the interior pocket can be a first interior pocket and the fastener can include a second interior pocket extending in an axial direction inside the shaft, radially inward from the land surface, and axially spaced apart from the first interior pocket by a solid internal wall of the shaft. In certain embodiments, the flexure opening can overlap axially with the first interior pocket but not with the second interior pocket.

In certain embodiments, the interior pocket can be a first interior pocket in a plurality of interior pockets spaced apart axially along the longitudinal axis within the shaft. The torque driver can be a first torque driver in a plurality of torque drivers each seated in respective one of the plurality of interior pockets. In certain such embodiments, the flexure opening can be a first flexure opening in a plurality of flexure openings each opening into and overlapping axially with a respective one of the plurality of interior pockets. In certain embodiments, each of the flexure openings can have a proximal end and a distal end, where the proximal ends of the flexure openings can all terminate at a first circumferential position (e.g., a clock position) relative to the longitudinal axis and the distal ends of the flexure openings can all terminate at a second circumferential position relative to the longitudinal axis.

In embodiments, an external thread can wind helically round the shaft. The land surface can be wound helically around the shaft axially offset from the external thread so that the land surface alternates with the external thread in an axial direction along the shaft. The external thread can extend in a radial direction beyond the land surface, and the flexure opening can extend helically in parallel with the external thread. In certain embodiments, the fastener may not include any external thread (e.g., such as for use as an intramedullary nail).

In embodiments, a head can be included at the proximal end of the shaft configured to engage a driver for turning the fastener about the longitudinal axis. The distal end of the shaft can define a narrowing tip that tapers down along the longitudinal axis in a distal direction. In certain embodiments, the narrowing tip can include a self-tapping recess or a self-drilling feature.

In certain embodiments, the fastener can be additively manufactured and the torque drivers can be captured inside the interior pockets. In certain embodiments, the fastener can be of a metallic material, such as titanium or stainless steel. In certain embodiments, the fastener can be of a biocompatible material or a combination of one or more biocompatible materials. In certain embodiments, the fastener can be configured to bend up to and beyond 45° off of the longitudinal axis. In embodiments, the fastener can be used for orthopedic applications. In certain embodiments, the orthopedic applications can include, vertebral spacers, acetabular cups, glenoid fossa prostheses, scaphoid prostheses, cervical spine implants, thoracic spine implants, lumbar spine implants, glenohumoral joint, hip joints, wrists, plates for both cervical and lumbar spine.

In accordance with at least one aspect of this disclosure, a flexible fastener includes a shaft body having a proximal end and a distal end spaced apart along a longitudinal axis, where the shaft body including a land winding helically around the shaft and having a first thickness than extends from an outer diameter of the shaft body radially inward to an inner diameter of the shaft body. A shaft core extends from the proximal end to the distal end, and the shaft core has a second thickness that extends from an outer diameter radially inward to an inner diameter. The shaft core is arranged within the land and spaced apart from the inner diameter of the shaft body such that an annular space is defined between the inner diameter of the land and an outer diameter of the shaft core. In certain embodiments, the central lumen is dimensioned to accommodate passage of a surgical instrument or guidewire therethrough, and/or promote flexure of the shaft.

A flexure opening extends through the land in the radial direction through the first thickness and extends helically about the longitudinal axis to provide for flexure of the shaft. A torque bridge extends from the inner diameter of the shaft body radially inward to the outer diameter of the shaft core for developing toque along the shaft body when a driving torque is applied to the proximal end of the shaft body. Further, a central lumen defined through the shaft core within the inner diameter of the shaft core, extending from the proximal end to the distal end.

The flexible fastener can include a plurality of torque bridges spaced apart helically about the longitudinal axis, where each torque bridge of the plurality of torque bridges are defined at a respective axial position adjacent the flexure opening. In certain embodiments, each torque bridge is positioned axially closer to a proximal flexure opening than to a center of the land in which the respective torque bridge is defined.

The proximal end of the shaft body further includes a driving head and a shaft neck extending distally therefrom, where the shaft neck tapers towards a threaded portion in a distal direction and the distal end further includes a narrowing tip. The threaded portion incudes a helical thread extending radially outward from the land. The flexure opening is defined in the threaded portion and extends from an axial position adjacent the shaft neck helically about the longitudinal axis to an axial position adjacent the narrowing tip as a single contiguous flexure opening. The flexure opening winds helically around the shaft axially offset from the external thread so that the flexure opening alternates with the external thread in an axial direction along the shaft. In certain embodiments, the flexure opening extends helically in parallel with the external thread. The shaft core extends from an axial position within the shaft neck, between the driving head and the threaded portion, along the longitudinal axis to the axial position adjacent the narrowing tip.

In certain embodiments, the plurality of torque bridges arranged in a line spaced apart helically beginning from the axial position within the shaft neck and terminating the axial position adjacent the narrowing tip. In certain embodiments, the torque bridges are integrally formed with the shaft and shaft core.

The fastener includes head at the proximal end of the shaft configured to engage a driver for turning the fastener about the longitudinal axis and the distal end of the shaft defines a narrowing tip that tapers down along the longitudinal axis in a distal direction. In certain embodiments, the narrowing tip can be or include a self-tapping or self-drilling feature.

In accordance with at least one aspect of this disclosure, an orthopedic implant system includes an implant configured to be fastened in place with a flexible fastener and an implant tool. In certain embodiments the implant system further includes a flexible fastener.

In certain embodiments, the implant includes an implant body having opposed superior and inferior faces configured for engaging the implant between a superior vertebra and an inferior vertebra, respectively, a bore defined through the implant body for receiving the flexible fastener, the bore extending through the implant body form a bore entrance defined in a first coronal face of the implant body and along a first axis to a bore exit defined in the superior or inferior face of the implant body and along a second axis that is angled with respect to the first axis to facilitate implantation of the implant, and a coupling region defined in the first coronal face configured to couple an implant tool to the implant during implantation of the implant. The implant tool can include a driver guide for receiving a driver, and a coupling region for mating with the coupling region of the implant.

The first coronal face extends between the superior and inferior faces, a second coronal face extends between the superior and inferior faces, opposite the first coronal face. In certain embodiments, the implant body can be trapezoidal. In certain embodiments, the implant body can include a lattice or matrix region for accommodating biomaterial (e.g., bone graft). The implant body can include a camming surface in the bore for turning the tip of the flexible fastener from the first axis to the second axis as the flexible fastener is advanced within the bore. In certain embodiments, the camming surface can turn the tip of the flexible fastener along the second axis, which can be angled with respect to the first axis at an angle in the range of about 30° to about 45°.

In certain embodiments, the driver guide can include a tubular casing extending proximally from the coupling region configured to align with the bore entrance of the implant with the implant tool coupled to the implant. The tubular casing can be threaded with interior threads configured to mate with exterior threads of a driver, and the threads of the tubular casing can be timed according to threads of the flexible fastener such that the driver and flexible fastener remain engaged as they are driven. The coupling region of the implant can include a threaded female portion, and the coupling region of the implant tool can include a threaded male portion configured to be threaded into the threaded female portion of the implant to couple the implant to the implant tool.

In certain embodiments, the bore is a first bore and the implant body can include a second bore therethrough having a bore entrance defined in the first coronal face and along a third axis parallel to the first axis and a bore exit defined in the superior face and along a fourth axis that is angled with respect to the third axis. A third bore can be defined in the implant body having a bore entrance defined in the first coronal face and along a fifth axis parallel to the first axis and a bore exit defined in the inferior face and along a sixth axis that is angled with respect to the fifth axis.

The implant tool can further include a plurality of driver guides, e.g., three driver guides, each driver guide configured to align with a respective bore entrance with the implant tool coupled to the implant, where each driver guide includes an elongated body defining a tubular casing for receiving a respective driver. In certain such embodiments, the driving guides and associated drivers can extend nearly parallel to one another for use through small surgical openings but can be angled slightly offset to one another to avoid interfering with driving operations thereof.

In accordance with at least one aspect of this disclosure, method of implanting an orthopedic implant includes the steps of, coupling an implant to an implant tool, inserting the implant into an orthopedic implant location, where the implant and implant tool include any one of the embodiments described herein, and affixing the implant to a bone by driving a flexible fastener in through the implant body, where the flexible fastener include any one of the embodiments described herein.

In certain embodiments, coupling the implant to the implant tool further includes, inserting a coupling rod into a coupling region of the implant tool such that the coupling rod extends beyond the coupling region of the implant tool and into a coupling region of the implant and threading the coupling rod into the coupling region of the implant. Driving the flexible fastener can further include inserting the flexible fastener and a driver into a driving guide of the implant tool and engaging the driver with a driving head of the flexible fastener within the implant to drive the flexible fastener through the implant and into bone tissue at the implant location. The method can further include removing the coupling rod from the implant to remove the implant tool from the implant after the implant is affixed to the implant location.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is a side elevation view of an embodiment of a bendable fastener constructed in accordance with the present disclosure, showing the thread, the land, and the flexure openings;

FIG. 2 is a cross-sectional plan view of the bendable fastener of FIG. 1, looking upward as oriented in FIG. 1, showing the torque drivers in the interior pockets;

FIG. 3 is a cross-sectional side elevation view of the bendable fastener of FIG. 1, showing the torque faces of the torque drivers not visible in FIG. 2;

FIG. 4 is a cross-sectional oblique elevation view of the bendable fastener of FIG. 1, showing the diagonal-cross sections of the torque drivers relative to the views of FIGS. 2 and 3;

FIG. 5 is an end elevation view of the head of the bendable fastener of FIG. 1, showing the angle of cross-section for FIG. 4;

FIG. 6 is a cross-sectional end elevation view of the bendable fastener of FIG. 1, showing the axial cross-section of one of the torque drivers at the position indicated in FIG. 2;

FIG. 7 is a cross-sectional end elevation view of the bendable fastener of FIG. 1, showing the axial cross-section through the narrowing neck of the toque driver;

FIG. 8 is a perspective view of the bendable fastener of FIG. 1, showing portions of the land that do not have flexure openings;

FIG. 9 is a perspective view of the bendable fastener of FIG. 1, showing the distal cul-de-sac shaped ends of the flexure openings, whereas the proximal cul-de-sac shaped ends are shown in FIG. 1;

FIG. 10 is a side elevation view of the torque driver of FIG. 4, showing some of the torque faces;

FIG. 10A is an enlarged exploded perspective view of a portion of FIG. 10, showing a torque driver exploded out of a respective interior pocket;

FIG. 11 is a schematic view of the bendable fastener of FIG. 1, showing a portion of thread, land, and flexure openings as though they are unwound from the helix, with angular locations as designated in FIG. 5 labeled;

FIG. 12 is a perspective view of the bendable fastener of FIG. 1, showing the fastener bending;

FIG. 13 is a side elevation view of an embodiment of another bendable fastener constructed in accordance with the present disclosure, showing a thread, a land, and a flexure opening;

FIG. 14 is a cross-sectional view of the bendable fastener of FIG. 13, taken along section 14-14, showing a shaft core, torque bridges, and a central lumen;

FIG. 15 is an enlarged, partial cross-sectional view of a proximal end of the bendable fastener of FIG. 14, showing an annular space defined between the shaft and shaft core,

FIG. 16 is an enlarged, partial cross-sectional view of a proximal end of the bendable fastener of FIG. 14, where the shaft core is shown removed for clarity,

FIG. 17 is a cross-sectional view of the bendable fastener of FIG. 13 taken along section 17-17, showing the showing the external threads, the shaft core, the torque bridges, and the central lumen;

FIG. 18 is a perspective view of the bendable fastener of FIG. 13 showing the bendable fastener bending;

FIG. 19 is a perspective view of an embodiment of an orthopedic implant system, including an orthopedic implant, a bendable fastener, and an implant tool, showing the system in an assembled state;

FIG. 20 is an exploded perspective view of the orthopedic implant system, showing the orthopedic implant and implant tool disassembled from one another;

FIG. 21 is a bottom perspective view of the orthopedic implant showing an inferior face of the implant;

FIG. 22 is an enlarged cross-sectional view of the implant of FIG. 21 taken along section 22-22, showing a bore defined in the implant body, the bore extending along a first axis and a second axis;

FIGS. 23-26 show an animation of the assembly of the orthopedic implant system of FIG. 19, showing the implant being coupled to the implant tool, where:

FIG. 23 shows the implant tool in isolation;

FIG. 24 shows the implant being coupled to the implant tool;

FIG. 25 shows rotation of a coupling rod of the implant tool to secure the implant to the implant tool; and

FIG. 26 shows removal of a driving knob from the coupling rod;

FIGS. 27-32 show an animation of a method for performing an orthopedic procedure, where:

FIG. 27 is a perspective view of the implant tool being inserted the implant into an implant location;

FIG. 28 is a perspective view of the implant inserted into the implant location with the implant tool still coupled thereto;

FIG. 29 is a perspective view showing a driver and bendable fastener being inserted into the implant tool for affixing the implant to the implant location;

FIG. 30 is an enlarged, partial cross-sectional view taken along the section 30-30, showing the driver and bendable fastener inserted into the implant tool;

FIG. 31 shows the cross-sectional view of FIG. 30 where the driver has advanced the bendable fastener through the implant and into the implant location; and

FIG. 32 is a perspective view of the implant location showing removal of the implant tool from the implanted implant;

FIG. 33 is a perspective view of an embodiment of another bendable fastener;

FIG. 34 is a perspective view of an embodiment of another bendable fastener shown installed in another implant;

FIG. 35 is a perspective view of an embodiment of another bendable fastener;

FIG. 36 is a perspective view of another orthopedic procedure, showing an embodiment of the bendable fastener being cannulated over a guidewire for placement in a metatarsal; and

FIG. 37 shows the bendable fastener fixed in the metatarsal with the guidewire removed, the bendable fastener following the curve of the metatarsal.

### DETAILED DESCRIPTION OF THE PREFERRED EMBODIMENTS

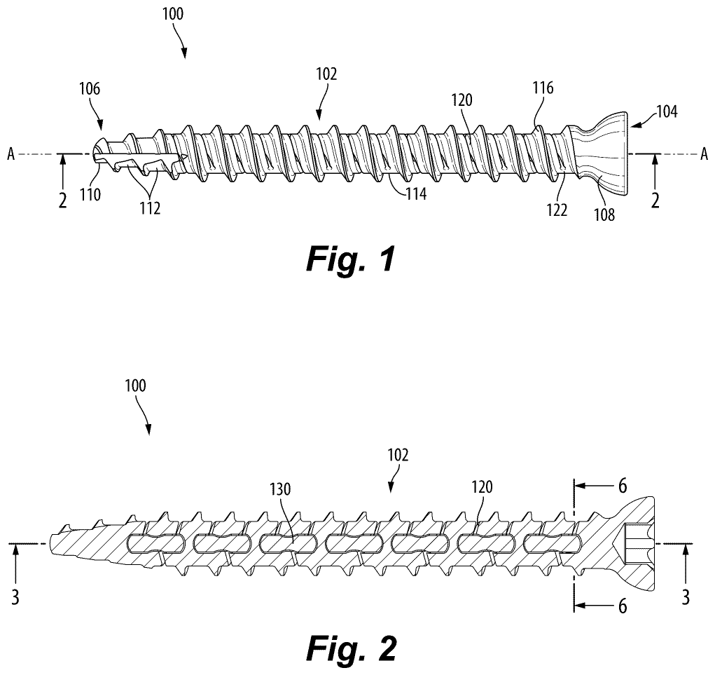

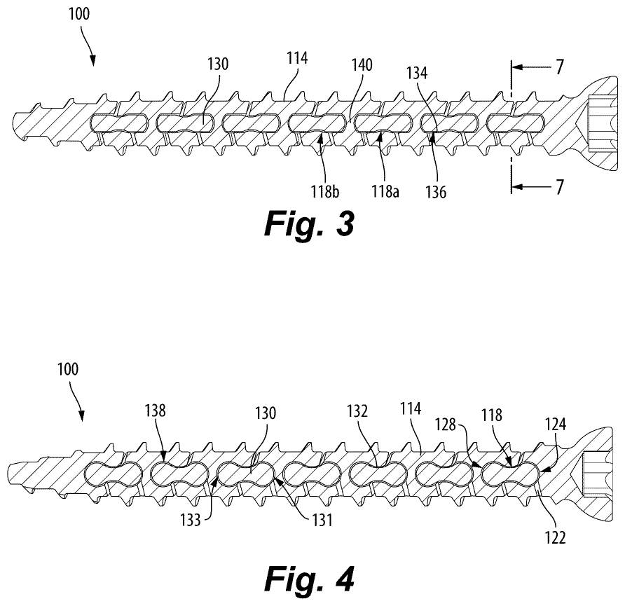

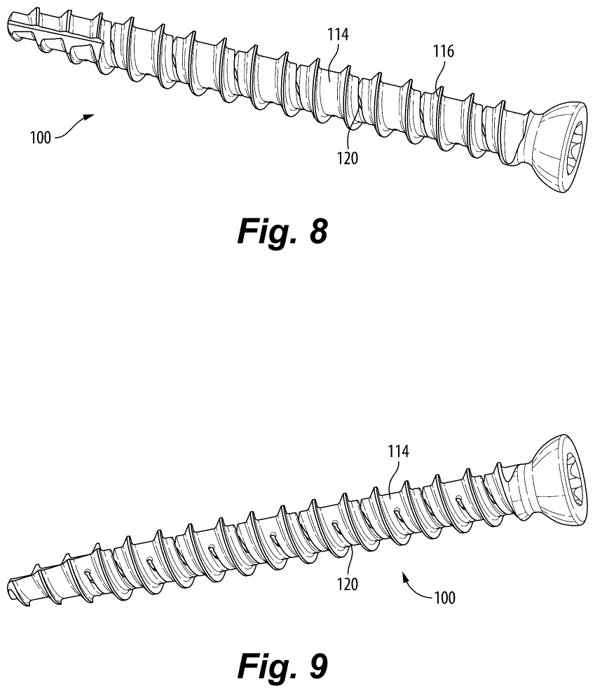

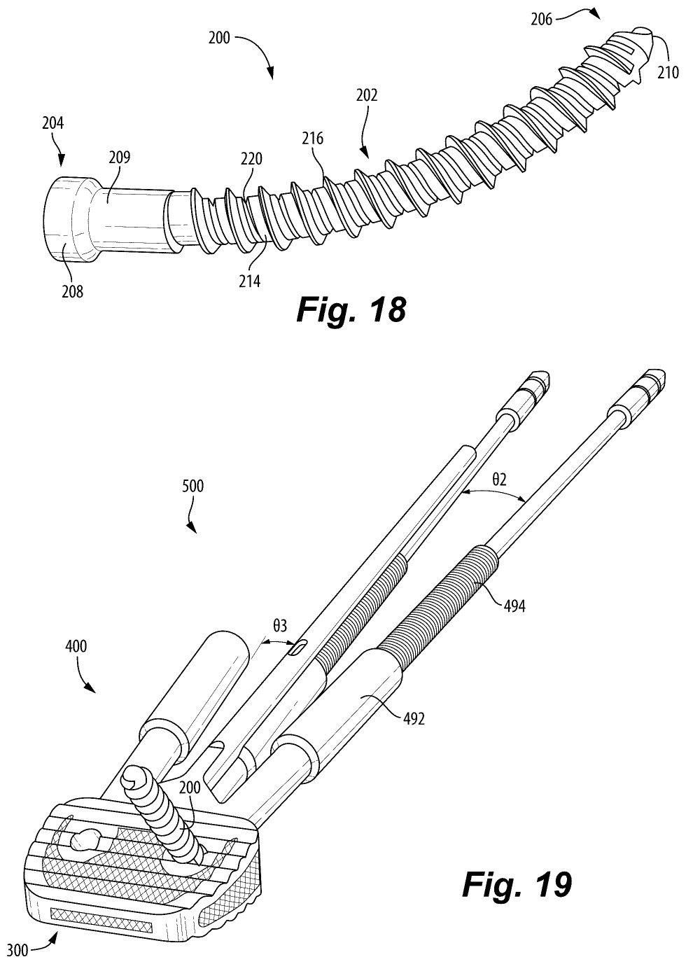

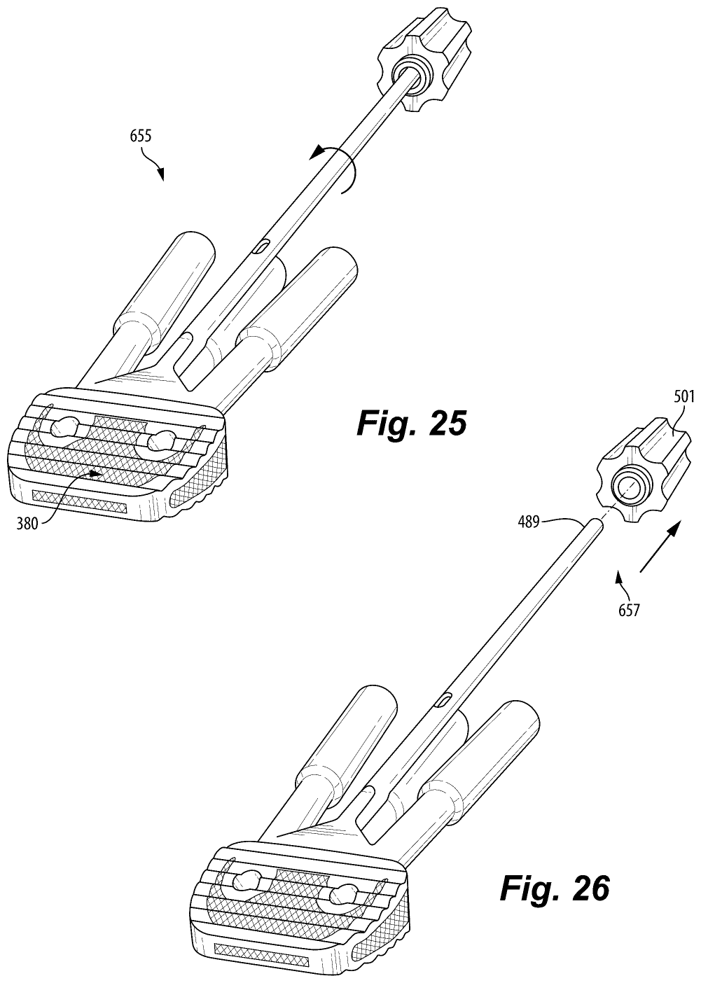

Reference will now be made to the drawings wherein like reference numerals identify similar structural features or aspects of the subject disclosure. For purposes of explanation and illustration, and not limitation, a partial view of an embodiment of a bendable fastener in accordance with the disclosure is shown in FIG. 1 and is designated generally by reference character 100. Other embodiments of systems in accordance with the disclosure, or aspects thereof, are provided in FIGS. 2-35, as will be described. The systems and methods described herein can be used to provide flexure of fasteners such as orthopedic screws and nails with improved strength and flexibility relative to traditional configurations.

With reference to FIGS. 1-4, in accordance with at least one aspect of this disclosure, a flexible (e.g., bendable) fastener 100 can include a shaft 102 having a proximal end 104 and a distal end 106 spaced apart along a longitudinal axis A. In embodiments, a head 108 can be included at the proximal end 104 of the shaft 102 configured to engage a driver (not shown) for turning the fastener 100 about the longitudinal axis A. The distal end 106 of the shaft can define a narrowing tip 110 that tapers down along the longitudinal axis A in a distal direction. In certain embodiments, the narrowing tip 110 can include a self-tapping recess 112. In certain embodiments, the narrowing tip 100 can include a self-drilling feature.

A land surface 114 can be wound helically around the shaft 102. In embodiments, an external thread 116 can wind helically round the shaft 102. The land surface 114 can be wound helically around the shaft 102 axially offset from the external thread 116 so that the land surface 114 alternates with the external thread 116 in an axial direction along the shaft 102. The external thread 116 can extend in a radial direction beyond the land surface 114. In certain embodiments, the fastener 100 may not include any external thread 116 (e.g., such as for use as an intramedullary nail). In embodiments, the land surface 114 and/or the external thread can wind about the shaft 102 at constant intervals or variable intervals, or both. In certain embodiments, the external thread 116 can be or include cortical (e.g. threads having a finer pitch) and/or a cancellous (e.g., threads having a larger pitch) style threads.

With reference to FIGS. 2-4 and 8-11, an interior pocket 118 can extend in an axial direction inside the shaft 102, radially inward from the land surface 114. A flexure 120 opening extends through the shaft 102 in the radial direction from the land surface 114 to an inward facing surface of the interior pocket 118. The flexure opening 120 can extend helically about the longitudinal axis A to provide for flexure of the shaft 102, of the land surface 114, and the external thread 116, and the flexure opening 120 can extend helically in parallel with the external thread 116. In embodiments, a proximal end 122 of the flexure opening 120 can be axially proximate a proximal end 124 of the interior pocket 118 with respect to the longitudinal axis A (e.g., as can be seen in FIG. 4).

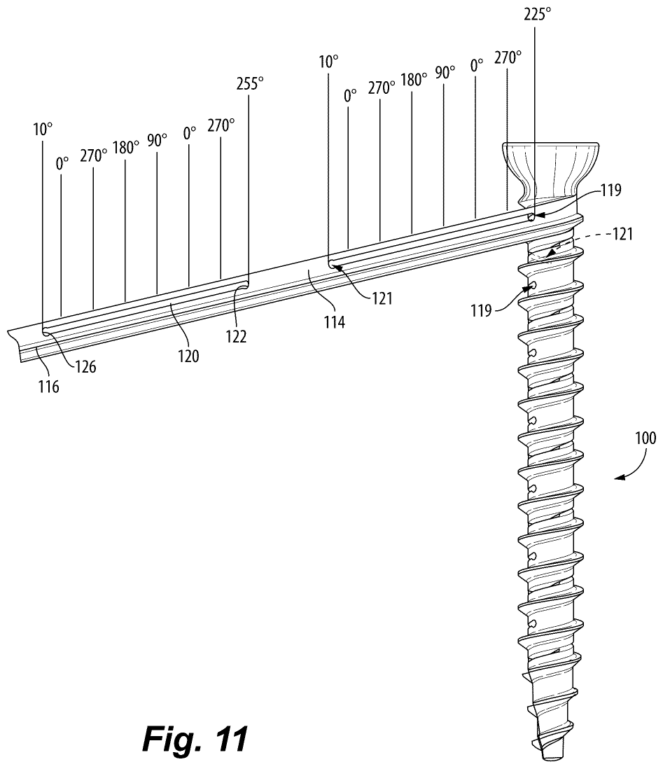

The flexure opening 120 can wind around the interior pocket 118 to a distal end 126 of the flexure opening 120 that is proximal to a distal end 128 of the interior pocket 118. In FIGS. 1-4, the distal end 126 of the flexure opening 120 is on a backside of the fastener 100 and is out of view. FIG. 9 shows a rotated perspective view wherein the distal end 126 of the flexure opening 120 is visible, but wherein the proximal end 122 of the flexure opening 120 is not visible. The “unwound” schematic view shown in FIG. 11 shows both the proximal 122 and distal 126 ends of the flexure opening 120. In embodiments, the distal end 126 of the flexure opening 120 can define a distal stress-reducing cul-de-sac shape, and the proximal end 122 of the flexure opening 120 can define a proximal stress-reducing cul-de-sac shape (e.g., as best seen in FIG. 11).

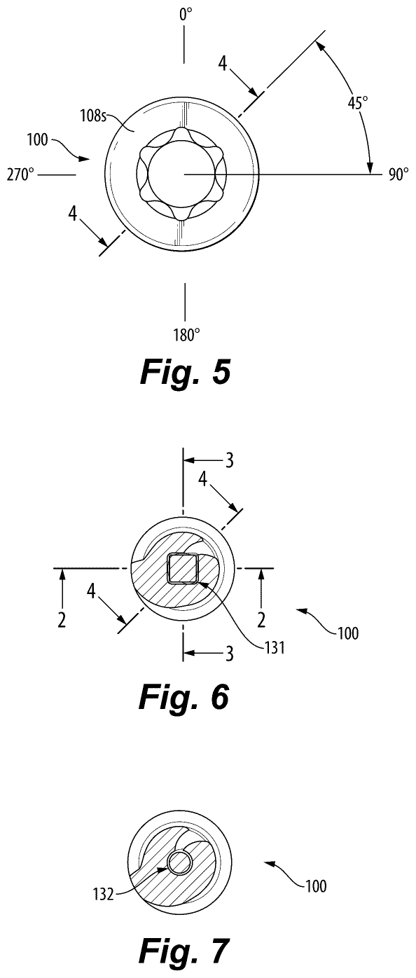

With further reference to FIG. 11, in certain embodiments, the flexure opening 120 can wrap beyond 360° around the interior pocket 118 circumferentially relative to the longitudinal axis A. For example, FIG. 11 shows a partially “unwound” fastener 100 where the proximal end 122 of the flexure opening 120 can begin at a first location 119 and the distal end 126 of the flexure opening 120 can terminate at a second location 121 on the fastener 100. In FIG. 5, a circumferential scale is defined looking down at the head 108 of the fastener as oriented in FIG. 5. This angular position scale is partially replicated on FIG. 1. In the view of FIG. 1, the circumferential position of 0° is the top of the fastener 100, 180° is the bottom, 90° is the side of the fastener facing into the page, and 270° is the side of the fastener facing out of the page. Transferring this scale to the view of FIG. 11, it can be seen that the first location 119 is at a position between 180° and 270°, but much closer to 270° than 180°. The first location 119 can be defined as 225° for example. The flexure opening 120 then extends around the shaft 102 (e.g., wrapping in a direction into the page), past 270° for a first time, past 0° for a first time, past 180°, past 90°, past 270° for a second time, past 0° for a second time, until termination at the second location 121 at some position between 0° and 90°, but much closer to 0° than 90°. The second location 121 can be defined as 10° on the circumferential scale. The circumferential scale is shown in a planar view on the unwound portion of FIG. 11.

With reference to FIGS. 1-7, 10, and 10A, a torque driver 130 is seated in the interior pocket 118. In certain embodiments, the flexure opening 120 can be too small to admit the torque driver 118 therethrough so the torque driver 130 is captured in the interior pocket 118. In embodiments, the torque driver 130 can be free floating within the interior pocket 118 (e.g., not integral with or affixed to) when the shaft 102 is in a relaxed state where no torque is applied to the shaft 102. In embodiments, the interior pocket can include a proximal portion 124 housing a proximal portion 131 of the torque driver 130 connected by a narrowing neck 129. The interior pocket 118 can also include a distal portion 128 housing a distal portion 133 of the torque driver 133. In embodiments, the interior pocket 118 can define a narrowing neck 132 connecting between the proximal and distal portions 124, 128 through which the narrowing neck 129 of the torque diver 130 passes.

The toque driver 130 can have a torque face 134 configured to abut a torque face 136 of the interior pocket 118 to develop toque along the shaft when a driving torque is applied to the proximal end 104 of the shaft 102. The torque face 134 of the torque driver 130 can be a first torque face 134a of the proximal portion 131 of the torque driver 130 and the proximal portion 131 of the torque driver 130 can include at least one additional torque face 134b, c, d. In embodiments, the distal portion 133 of the torque driver 130 can include a plurality of torque faces 134, e.g., at least 134e, f, g. Each of the torque faces 134 of the torque driver can face a corresponding torque face 136 of the interior pocket 118 for abutment and for engaging the corresponding torque face 136 when torque is applied to the head 108 to drive the torque from the proximal end 104 to the distal end 106 of the shaft 102.

In embodiments, a clearance gap 138 can be defined between the toque driver 130 and the interior pocket 118 so the toque driver 130 can move (e.g. by gravity as a user moves the fastener through a space such as an operating room) within the interior pocket 118 as long as the shaft 102 is in the relaxed state. The clearance gap 138, torque faces 134 of the torque driver 130, and the torque faces 136 of the interior pocket 118 can be configured so the clearance gap 138 is too small to allow the rotation of the torque driver 130 about the longitudinal axis A beyond a point where the torque faces 134, 136 of the interior pocket 118 and of the torque driver 130 come into abutment. The distal and proximal portions 131, 133 of the torque driver 130 can define a square axial cross-section with a diagonal too large to rotate within the interior pocket 118 beyond abutment of the torque faces 134, 136 (e.g., as shown in FIGS. 2, 3, and 6). In certain embodiments, abutment (e.g., as used herein) may not necessarily require full planar contact or engagement between faces. The narrowing neck 132 of the torque driver can define a circular axial cross-section (e.g., as shown in FIG. 7).

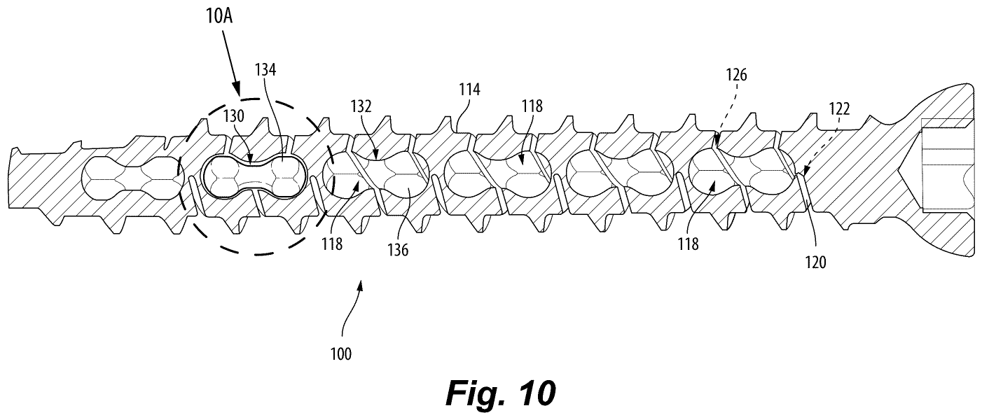

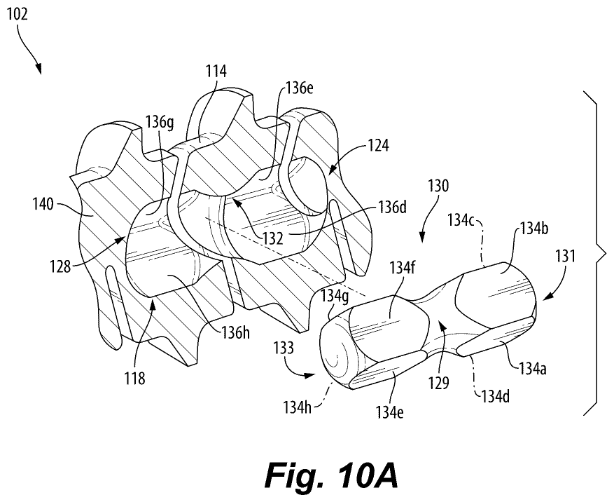

As can be seen most clearly in FIGS. 4 and 10, in embodiments, the proximal and distal portions 131, 133 of the torque driver 130 can have a cul-de-sac cross-sectional shapes in a plane of the diagonal. In certain embodiments, the proximal and distal portions 131, 133 of the torque driver 130 can be aligned along the same diagonal. In certain embodiments the proximal and distal portions 131, 133 of the torque driver 130 can be clocked about the longitudinal axis A relative to one another.

In embodiments, e.g., as shown, the interior pocket 118 can be a first interior pocket 118a and the fastener 100 can include at least one additional interior pocket 118b extending in an axial direction inside the shaft 102, radially inward from the land surface 114, and axially spaced apart from the first interior pocket 118 by a solid internal wall 140 of the shaft 102. In certain embodiments, the flexure opening 120 can overlap axially with the first interior pocket 118a but not with the second interior pocket 118b. Each interior pocket 118 can include a respective torque driver 130 as described above. The flexure opening 120 can be a first flexure opening in a plurality of flexure openings each opening into and overlapping axially with a respective one of the plurality of interior pockets 118. In certain embodiments, each of the flexure openings 120 can have a proximal end 122 and a distal end 126, where the proximal ends 122 of the flexure openings 120 can all terminate at a first circumferential position (e.g., a clock position) relative to the longitudinal axis A and the distal ends 126 of the flexure openings can all terminate at a second circumferential position relative to the longitudinal axis A. For example, as shown in FIGS. 1, and 11, the proximal ends 122 of the flexure openings 120 are shown at the first circumferential position, and FIG. 9 shows the distal ends 126 of the flexure openings 120 at the second circumferential locations.

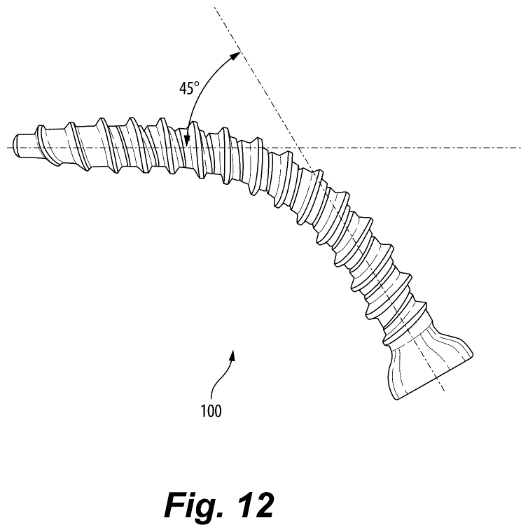

In certain embodiments, the fastener 100 can be additively manufactured and the torque drivers 130 can be captured inside the interior pockets 118. In certain embodiments, the fastener 100 can be of titanium and can be additively manufactured from titanium. In certain embodiments, the fastener 100 can be of a biocompatible material or a combination of one or more biocompatible materials. In certain embodiments, the fastener 100 can be configured to bend up to and beyond 45° off of the longitudinal axis A (e.g., as shown in FIG. 12). In embodiments, the fastener 100 can be used for surgical or orthopedic applications, for example, vertebral spacers, acetabular cups, glenoid fossa prostheses, scaphoid prostheses, cervical spine implants, thoracic spine implants lumbar spine implants, glenohumoral joint, hip joints, wrists, or the like. U.S. Pat. No. 9,597,199 to Glazer, which is herein incorporated by reference in its entirety, describes embodiments of fasteners for various implants.

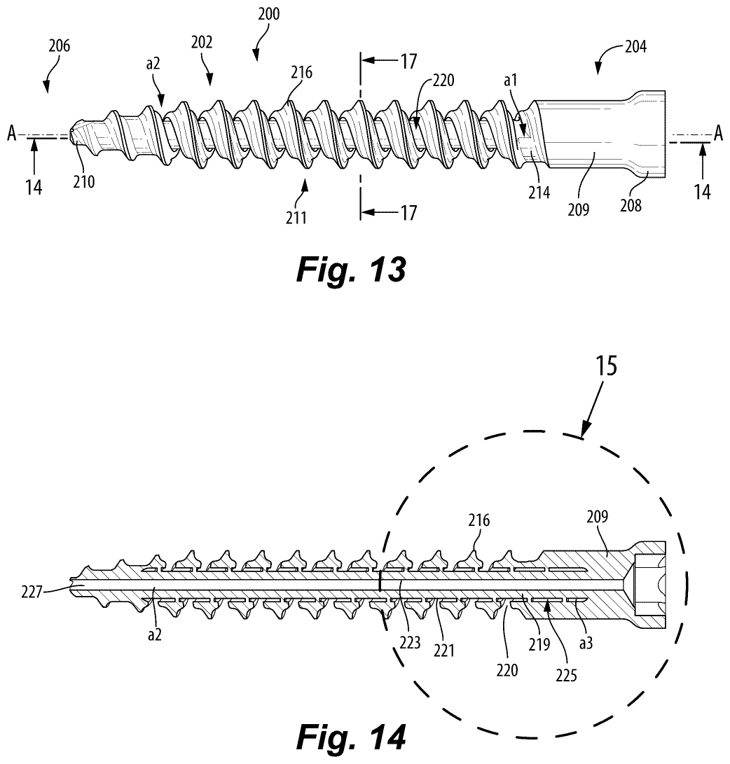

Turning now to FIGS. 13-18, another embodiment of a bendable (e.g., flexible) fastener 200 is shown. In FIGS. 19-32, the flexible fastener 200 is shown in use with an orthopedic implant system 500. The flexible fastener 200 can be similar to bendable fastener 100 and can have similar components and features with respect to flexible fastener 100. For brevity, the description of common elements that have been described above for flexible fastener 100 will not necessarily be repeated with respect to flexible fastener 200 as shown in FIGS. 13-18. The flexible fastener 200 as shown includes a cannulized flexible fastener for use in a variety of orthopedic applications.

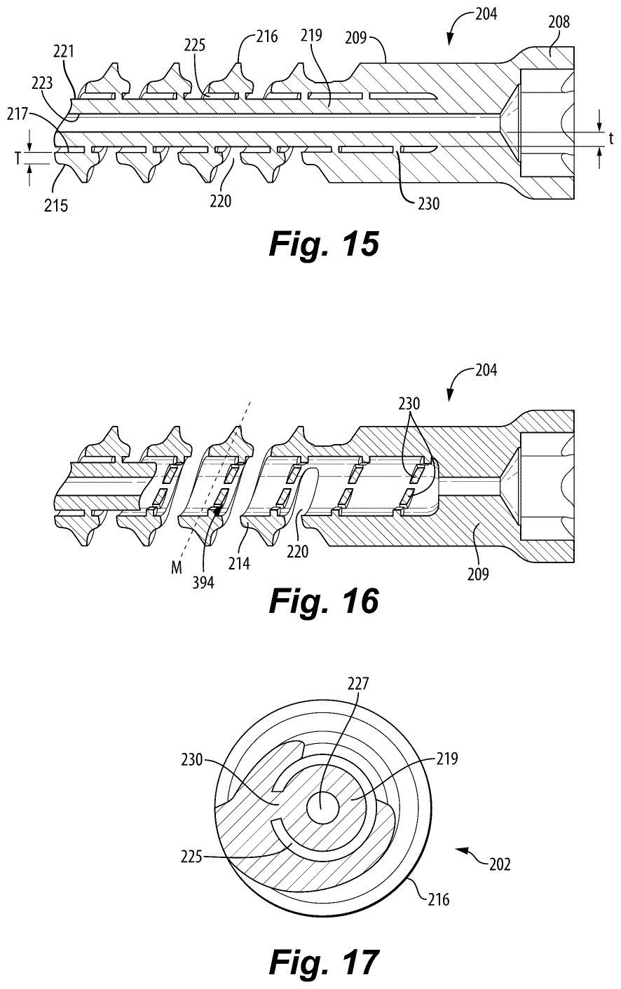

As shown in FIG. 13, the flexible fastener 200 includes a shaft body 202 having a proximal end 204 and a distal end 206 spaced apart along a longitudinal axis A. The proximal end 204 includes the driving head 208 and a shaft neck 209, which extends distally from the driving head 208 and tapers into a threaded portion 211. The land 214 winds helically around the shaft 202 and has a first thickness T that extends from an outer diameter 215 of the shaft body 202 radially inward to an inner diameter 217 of the shaft body 202. The thickness of the land 214 can best be seen in FIGS. 14 and 15.

The flexure opening 220 is defined in the threaded portion 211 and extends from an axial position a1 adjacent the shaft neck 209 helically about the longitudinal axis A to an axial position a2 adjacent the narrowing tip 210. The flexure opening 220 winds helically around the shaft 202 axially offset from the external thread 216 so that the flexure opening 220 alternates with the external thread 216 in an axial direction along the shaft 202, and where the flexure opening 220 extends helically in parallel with the external thread 216. The flexure opening 220 extends through the land 214 in the radial direction through the first thickness T and extends helically about the longitudinal axis A to provide for flexure of the shaft 202 as a single contiguous opening, which is different than fastener 100.

With reference now to FIG. 14, a shaft core 219 extends from the proximal end 204 to the distal end 206 of the shaft body 202. The shaft core 219 has a second thickness t that extends from an outer diameter 221 radially inward to an inner diameter 223. The shaft core 219 is arranged within the land 214 and spaced apart from the inner diameter 217 of the shaft body 102 such that an annular space 225 is defined between the inner diameter 217 of the land and an outer diameter 223 of the shaft core 219. This is more clearly seen in the enlarged view shown in FIG. 15 for example. Also shown in FIG. 14, a central lumen 227 is defined through the shaft core 219 and can be dimensioned to accommodate passage of a surgical instrument or guidewire therethrough, and/or promote flexure of the shaft 202. The shaft core 219 extends from an axial position a3 within the shaft neck 209, between the driving head 208 and the threaded portion 211, along the longitudinal axis A to the axial position a2 adjacent the narrowing tip 210.

Referring now to FIG. 16, a plurality of torque bridges 230 are included, extending from the inner diameter 217 of the shaft body 202 radially inward to the outer diameter 221 of the shaft core 219 for developing toque along the shaft body 202 when a driving torque is applied to the driving head 208. Since the fastener 200 includes the central lumen 227, the torque drivers 130 as shown in fastener 100 are omitted from the shaft 202 altogether, and the torque bridges 230 assume the function of transferring the torque from the land 214 to the shaft core 219 so that the shaft 202 does not unwind or tighten down on itself when being driven.

As can best be seen in FIG. 16, the torque bridges 230 are spaced apart helically about the longitudinal axis A. The torque bridges 230 are defined at an axial position a4 adjacent the flexure opening 220, specifically adjacent a proximal flexure opening with respect to the land 214 in which the bridge 230 is defined. For example, each torque bridge 230 can be positioned axially closer to a proximal flexure opening 220 than to a center (represented by exemplary midline M) of the land 214 in which the respective torque bridge 230 is defined, when viewed in a longitudinal cross-section (e.g., as shown in FIG. 16). The torque bridges 230 begin at the axial position a3 within the shaft neck 209 and terminate the axial position a2 adjacent the narrowing tip 210, or in other words, the torque bridges 230 start at an axial position closer to the driving head 208 than to the start of the external threads 216 or the start of the flexure opening 220. In certain embodiments, the torque bridges 230 are integrally formed with the shaft 202 and shaft core 219, where the shaft 202, shaft core 219, and torque bridges 230 are formed as a single unitary piece.

Certain applications of the bendable fastener 200, such as for use in interbody devices, can require more bending than conventional fasteners. To achieve this, bendable fasteners 200 as disclosed herein take into account the tradeoff between Second Polar Moment of Area J (mm⁴) and Area Moment of Inertia I (mm⁴), as described next. A higher polar moment of inertia J increases torsional strength and rigidity while reducing the maximum shear stress for a given torque. This allows the fastener 200 to withstand greater twisting forces without yielding. For a cannulated fastener, such as described herein, a larger outer radius or smaller inner radius increases J enhancing torsional strength. A smaller area movement of inertia I reduces bending stiffness, making the flexible fastener 200 more flexible under bending loads. This allows greater deflection without exceeding the material's yield strength. For a cannulated fastener, such as described herein, a smaller outer diameter or larger inner diameter lowers I, increasing bending flexibility.

Accordingly, the trade-off is that both I and J depend on the same geometric terms. So, for any given cannulated fastener (e.g., a conventional cannulated fastener) the cannula diameter and the outer diameter, increasing J for torsion (e.g., larger outer diameter) tends to increase I, reducing bending flexibility. A high J allows for better torsional properties, while a low I makes the fastener more bendable. Since these two properties are in conflict with each other In order to make a cannulated fastener bendable (e.g., as shown herein with respect to fastener 200) J and I need to be balanced in order to provide for appropriate bending while maintaining an acceptable torsional strength. Embodiments take this balancing act into consideration by maximizing J while minimizing I.

This becomes important when configuring the bendable fastener 200, for example, since it includes the shaft core 219, which is unique compared to conventional cannulated fasteners. Adding in the shaft core 219, requires adjusting the geometry of the cannula diameter 223 of the fastener 200 where the area varies down the fastener axis A. It can be difficult to achieve an optimal J:I ratio, but by changing the geometry internally, such as by including cannula 227 and the shaft core 219 core with the discreet connectors (e.g., torque bridges 230), the fastener 200 can achieve a non-circular cross section, allowing the fastener 200 to have a J:I ratio close to or within the range of 3-4. This ratio range allows for improved torsional strength while keeping desired flexibility. This ratio leverages non-circularity to exceed the circular limit (J/I=2), balances practical constraints (e.g. size, stress, manufacturability) for use as an orthopedic screw.

In certain embodiments, the flexible fastener 200 is configured and adapted so that both J and I are less than J and I of a solid fastener or conventional cannulated fastener. Since the flexible fastener 200 can be used in a variety of applications and may come in a variety of sizes, the outer diameter 215 and cannula diameter 223 can be selected according to a determined ratio, for example, in certain embodiments, a ratio of J:I can be between 3 and 4, and in certain embodiments, about 3.22.

With reference now to FIGS. 19-32, an embodiment of an orthopedic implant system 500 is shown. The orthopedic implant system 500 includes a flexible fastener (e.g., flexible fastener 200), an implant 300 (e.g., configured to be fastened in place with the flexible fastener described herein), and an implant tool 400. While FIGS. 19-32 are shown and described with respect to the flexible fastener 200, it should be appreciated by those skilled in the art having the benefit of this disclosure, that the orthopedic implant system 500 can be used with any suitable embodiment of flexible fastener as described herein.

In certain embodiments, the implant 300 can be an intervertebral implant for example configured for use as an intervertebral spacer in a lumbar interbody fusion procedure. While the implant 300 and implant tool 400 as described herein are described with respect to an anterior lumbar interbody fusion (ALIF), one having ordinary skill in the art given the benefit of this disclosure, would readily appreciate that the implant 300 and implant tool 400 can be modified for use with other interbody fusion procedures, such as posterior lumbar interbody fusion (PLIF), transforaminal lumbar interbody fusion (TLIF), lateral approaches (e.g., as shown in FIG. 34) such as oblique lateral interbody fusion (OLIF) and extreme lateral interbody fusion (XLIF), an Interfixated Cervical Interbody, or the like.

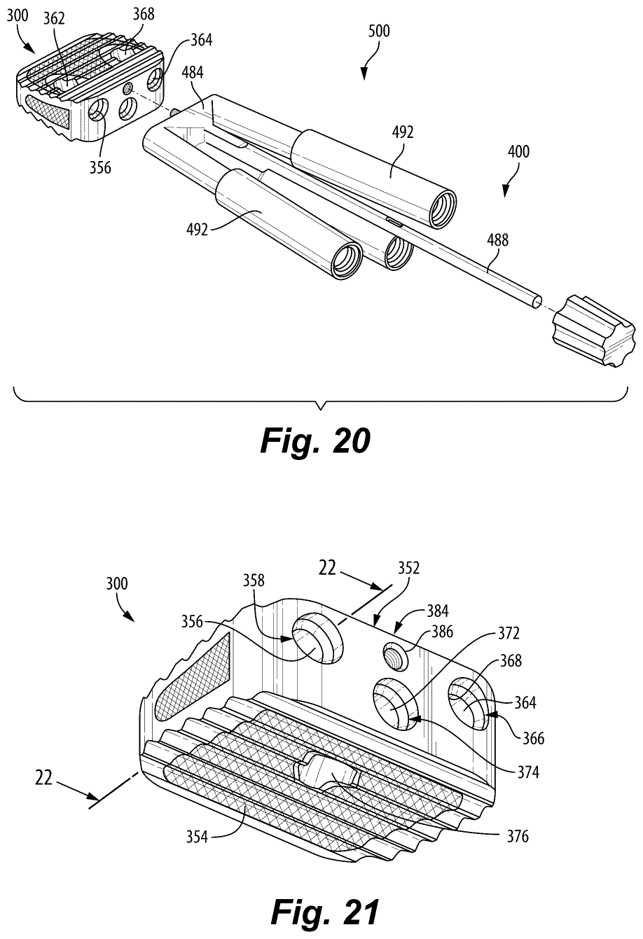

Withs specific reference to FIGS. 20-22, the implant 300 includes an implant body 350 having opposed superior 352 and inferior faces 354 configured for engaging the implant 300 between a superior vertebra 52 and an inferior vertebra 54, respectively. A first bore 356 is defined through the implant body 350 for receiving the flexible fastener 200, the bore 356 extending through the implant body 352 form a bore entrance 358 defined in a first coronal face 360 of the implant body 350 and along a first axis B to a bore exit 362 defined in the superior 352 or inferior face 354 of the implant body 352 and along a second axis C that is angled with respect to the first axis B. Any suitable number of bores 356 can be included in the implant body, depending on the procedure in which the implant is being used. In the example shown here, a total of three bores are shown.

A second bore 364 is defined in the implant body 350 having a bore entrance 366 defined in the first coronal face 350 and along a third axis D, parallel to the first axis B, and a bore exit 370 defined in the superior face 352 and along a fourth axis E that is angled with respect to the third axis C. A third bore 372 is defined in the implant body 350 having a bore entrance 374 defined in the first coronal face 360 and along a fifth axis F, parallel to the first axis B, and a bore exit 376 defined in the inferior face 354 and along a sixth axis G that is angled with respect to the fifth axis F. The orientation and relative positioning of the implant 300 to the implantation site is more clearly shown in FIGS. 28-32.

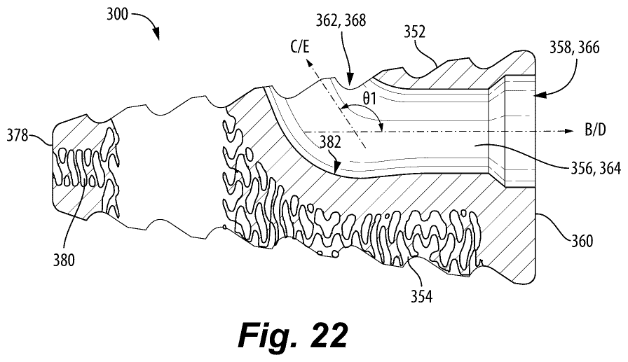

Still with reference to FIGS. 20-22, the first coronal face extends 360 between the superior 352 and inferior faces 354 and a second coronal face 378 extends between the superior 352 and inferior 354 faces, opposite the first coronal face 360. In certain embodiments, as shown, the implant body 350 can be trapezoidal and have a wedge-shaped profile. In certain embodiments, the implant body 350 can also include a lattice or matrix region 380 for accommodating biomaterial (e.g., bone graft). The implant body 350 can include a camming surface 382 in each respective bore 356, 356, 372 for turning the tip 210 of the flexible fastener 200 from the first/third/fifth axis B/D/F to the second/fourth/sixth axis C/E/G as the flexible fastener 200 is advanced within the respective bore 356, 356, 372. In certain embodiments, the second/fourth/sixth axis C/E/G can be angled with respect to the first/third/fifth axis B/D/F at an angle θ1, which can be about 30° to about 45°. This is best seen in FIG. 22, for example.

Referring now to FIGS. 19-25, a coupling region 384 is defined in the first coronal face 360 of the implant body 350 configured to couple an implant tool (e.g., tool 400) to the implant 300 during implantation of the implant 300. The implant tool 400 includes its own coupling region 484 for mating with the coupling region 384 of the implant 300. The coupling region 384 of the implant 300 can include a threaded female portion 386, and the coupling region 484 of the implant tool 400 can include a threaded male portion 486 configured to be threaded into the threaded female portion 386 of the implant 300 to couple the implant 300 to the implant tool 400. A coupling rod 488 is used to couple the implant 300 to the implant tool 400. With the coupling rod 488 inserted into the implant tool 400, the male threaded end 486 will extend beyond a distal face 490 of coupling region 484 of the implant tool 400, to be inserted into the threaded female portion 386 of the implant 300.

The implant tool 400 also includes a driver guide 492 for receiving an associated driver 494. The number of driver guides 492 and drivers 494 included on and with the tool 400 corresponds to a number of bores defined in the implant 300, which is determined based on the type of procedure being performed, and in certain instances, can be based on the size of the implant 300. In the embodiment shown herein, the implant tool 400 includes three driver guides 492 and three drivers 494, since three bores 356, 356, 372 are defined in the implant 300.

As shown, the respective driver guides 492 can include a tubular casing 496 extending proximally from the coupling region 484 configured to align with the respective bore entrance of the implant 300 when the implant tool 400 is coupled to the implant 300. The tubular casing 496 can be threaded with interior threads 498 configured to mate with exterior threads 499 of a respective driver 494. The threads 498 of the tubular casing 496 can be timed according to threads 216 of the flexible fastener 200 such that the driver 494 and flexible fastener 200 remain engaged as they are driven. The driver guides 492 and associated drivers 494 can extend nearly parallel to one another for use through small surgical openings but can be angled slightly offset to one another to avoid interfering with driving operations thereof, e.g., as shown by angles θ2, θ3. The orthopedic implant system 500 can further include a driving knob 501 for selectively coupling with the coupling rod 488 and each driver 494 for driving the coupling rod 488 and the drivers 494, in turn driving the flexible fastener 200 through the implant 300 and into bone 52, 54. The driving knob 501 can be coupled to and removed from each of the coupling rod 488 and the respective drivers 494 as needed depending on which component is being driven at a given time.

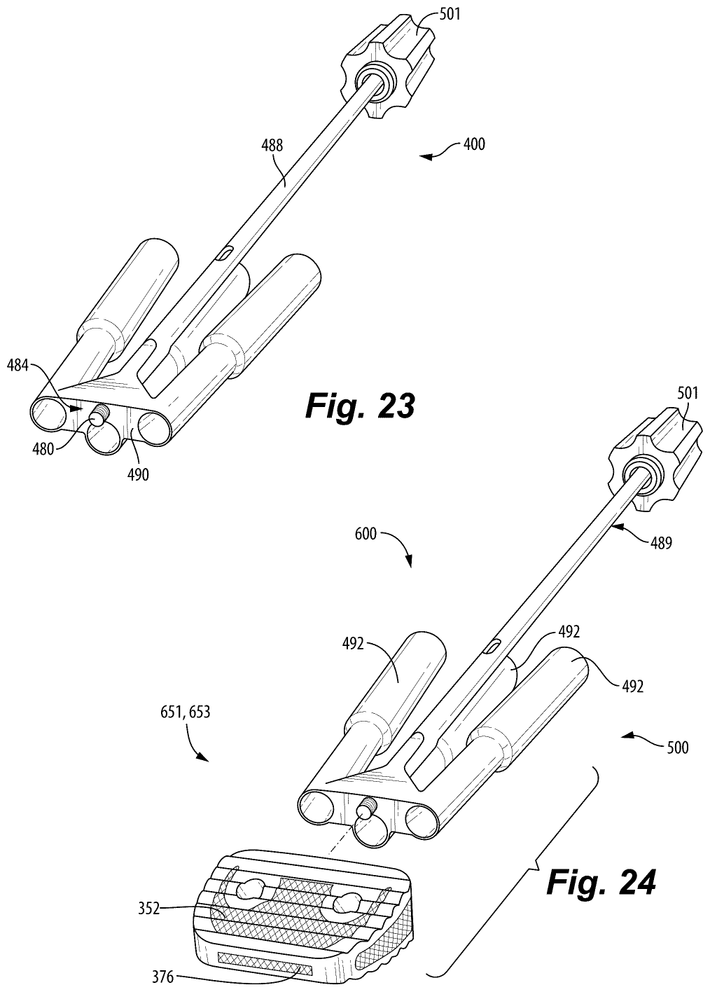

With reference now to FIGS. 23-32, and in accordance with at least one aspect of this disclosure, method 600 of implanting an orthopedic implant (e.g., implant 300) is shown. The method shown herein is referred to an ALIF procedure, an interior lumbar interbody fusion, though as noted herein, this specific procedure is only one example of a procedure shown for illustration, and not limitation.

The method 600 includes coupling 651 an implant (e.g., implant 300) to an implant tool (e.g., implant tool 400). Coupling 651 the implant 300 to the implant tool 400 includes inserting 653 the coupling rod 488 into the implant tool 400 such that the threaded male portion 486 of the coupling rod 488 extends beyond the distal face 490 of the coupling region 484 and inserting the threaded male portion 486 into the threaded female portion 386 of the implant 300. The driving knob 501 can then be attached to the proximal end 489 of the coupling rod for threading 655 the coupling rod into the implant, locking it in place for insertion at the implant location. The driving knob 501 can then be removed 657 from the coupling rod to be used later in the procedure. Coupling the implant tool to the implant is shown in the animation of FIGS. 23-26.

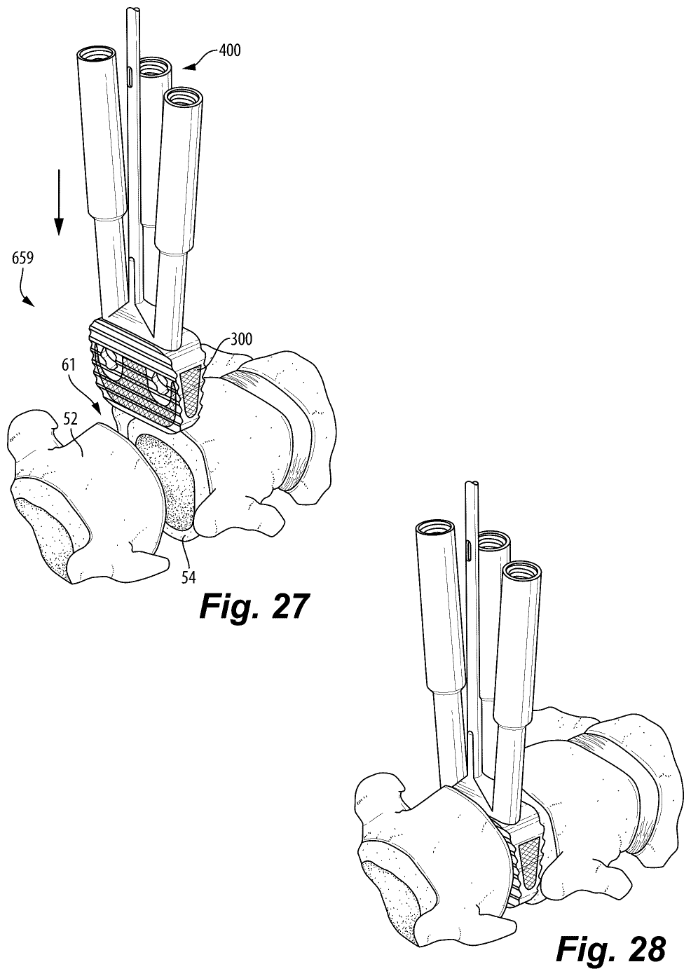

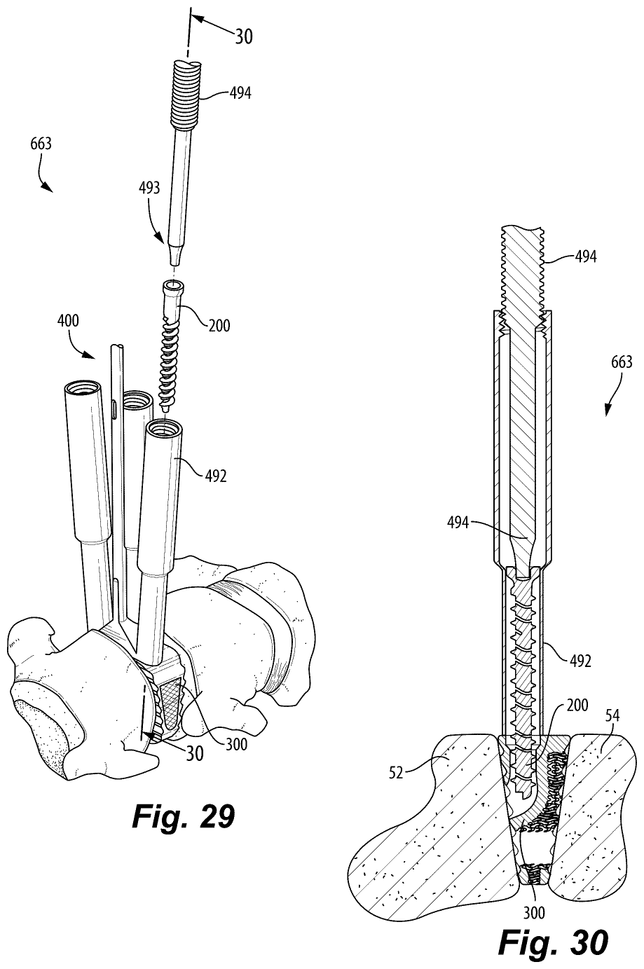

Next, the method 600 can include inserting 659 the implant into an orthopedic implant location 61 with the implant 300 coupled to the implant tool 400. As shown in FIGS. 27 and 28, the implant 300 is inserted into the implant location 61, in this example, between the vertebrae 52, 54. With the implant 300 seated in the proper location, the method 600 next includes the step of affixing 663 the implant to a bone (e.g., into the superior 52 and inferior vertebrae 54) by driving a flexible fastener (e.g., flexible fastener 200) in through the implant body 350 using the bores defined in the implant body, as shown in the animation of FIGS. 21-31. To perform the affixing step 663, a flexible fastener 200 is coupled to the driving end 493 of a respective driver 494, and the pair is inserted into a respective driver guide 492 in the implant tool 400. This is shown in FIG. 29.

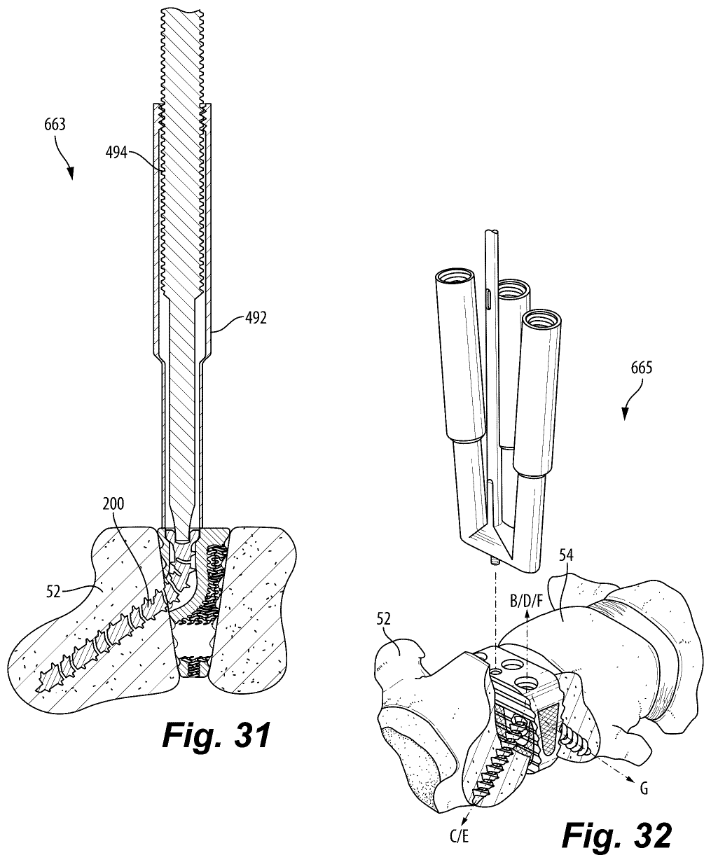

As shown in FIGS. 30 and 31, the driver 494 is threaded through the driving guide 492, whereupon when the distal tip 210 of the flexible fastener 200 hit the camming surface 382 in the bore of the implant body 350, the distal end 206 flexible fastener 200 is deflected at an angle (e.g., θ1) to extend out of the implant 300 through the respective bore exit. The driver 494 is rotated using the driving knob 501 removed from the coupling rod 488 in the prior step. Once the flexible fastener 200 is affixed in place, the respective driver 494 remains in its driver guide 492 until the end of the procedure. The next flexible fastener 200 is inserted into its bore in the same manner, and this process is repeated until all flexible fasteners 200 are in place. In this example, two flexible fasteners 200 will exit the implant 300 and insert into the superior vertebra 52, and one flexible fastener 200 will exit the implant 300 and insert into the inferior vertebra 54. Other procedures may result in a different fastener configuration. The driving knob 501 is passed between the drivers 494 during the duration of the operation as needed based on which driver 494 is being driven.

After affixing the implant 300 into the implant location 61 with the flexible fasteners 200, as shown in FIG. 32, the driving knob 501 is returned to the coupling rod 488 and the coupling rod 488 is rotated to back the coupling rod 488 out of the female portion 386 of the implant 300, removing 665 the implant tool 400, including all associated drivers 494 still within their driver guides 492, from the implant 300 all at once, while the implant 300 remains affixed in the implant location 61.

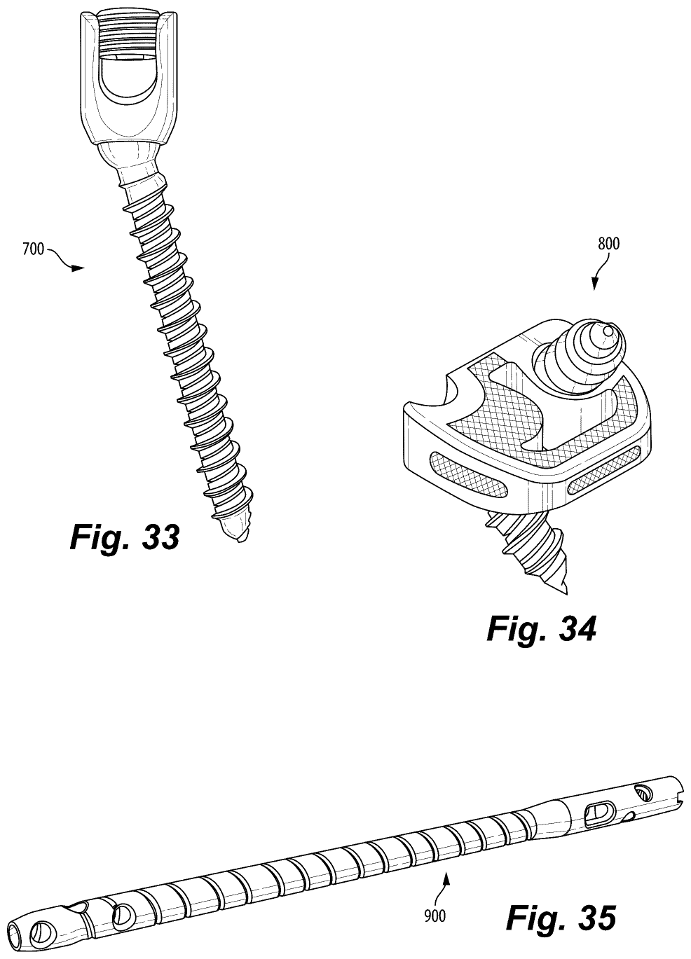

FIGS. 33-35 show additional embodiments of flexible fasteners for use in other various orthopedic applications. For example, FIG. 33 show a flexible fastener 700 configured and adapted for use with or as a pedicle screw, FIG. 34 shows a flexible fastener 800 configured and adapted for use with a cervical implant or for fixation through a laterally placed spacer, and FIG. 35 shows a flexible fastener 900 configured and adapted for use with or as an intramedullary nail or rod. Embodiments of the flexible fasteners 100, 200 described herein can be configured and adapted for additional uses not shown, for example, orthopedic instrumentation, trauma screws, cervical interbody devices, and alternative LIF approaches (e.g., other than anterior approach) as discussed hereinabove.

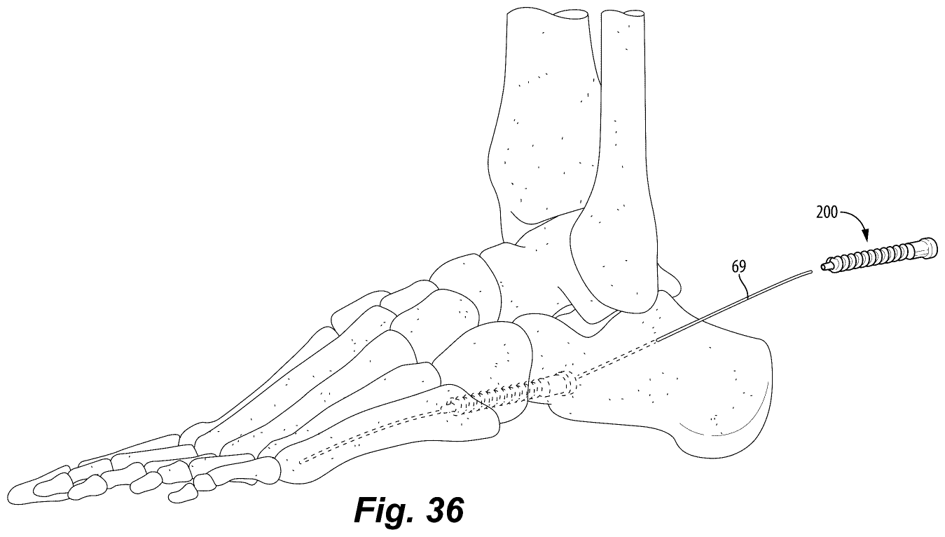

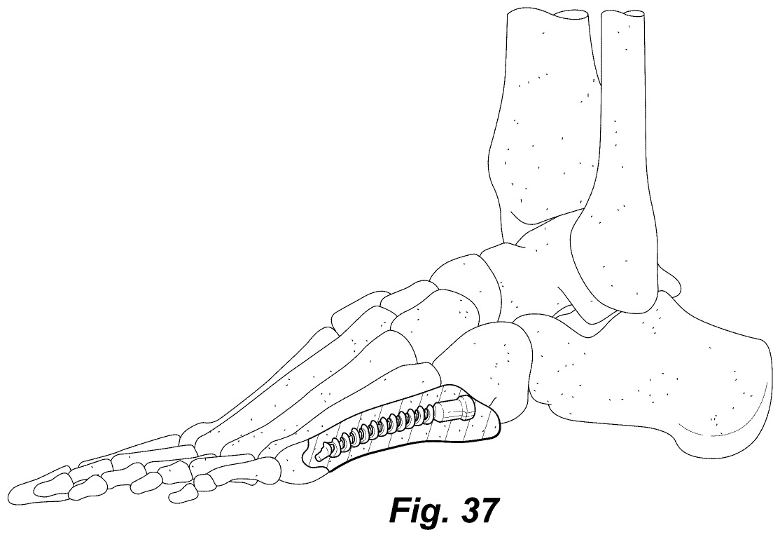

FIGS. 36 and 37 show another example of a surgical procedure in which the flexible fastener 200 can be employed. In this example, the flexible fastener 200 is shown in a repair of a fifth metatarsal fracture (e.g., a Jones fracture). Typically, the fifth metatarsal is curved, for example towards the outer edge of the foot and/or along the arch of the foot. Placing a rigid fastener into the curved bone can cause the rigid fastener to extend into the periosteum or even externalize through the metatarsal shaft or head. To combat this risk, the flexible fastener 200 is configured and adapted to bend and follow the curve of the metatarsal when being inserted. Similar risks can arise when repairing fractures of the metacarpals. Accordingly, it should be appreciated by those skilled in the art in view of this disclosure, the flexible fastener 200 can be used in such metacarpal repairs, though not shown herein.

In FIG. 36, a guidewire 69 (e.g., a K-wire) is shown inserted into the patient's foot and passing along and through the medullary canal of the fractured fifth metatarsal. The guidewire 69 can be inserted using conventional techniques, for example fluoroscopy. The flexible fastener 200 is shown in space, before being placed onto the guidewire 69. A phantom fastener 200 is shown farther down the guidewire, cannulated over the wire to show a trajectory of the fastener 200 as the procedure progresses. To perform the repair, the fastener 200 is placed over the guidewire 69 and then driven into place, following the along the guidewire 69 to the position where the guidewire 69 begins to curve within the fifth metatarsal. At this point, the fastener 200 bends along the curve of the medullary canal and is eventually driven fully into the medullary canal of the fifth metatarsal, following its natural curve. Fluoroscopy is performed throughout placement of the fastener 200 to ensure proper bending radius and alignment within the medullary canal. FIG. 37 shows the fastener 200 fixed in the bone in a completed repair, with the guidewire 69 removed.

The methods and systems of the present disclosure, as described above and shown in the drawings, provide for flexure of fasteners such as orthopedic screws and nails with improved strength and flexibility relative to traditional configurations. While the apparatus and methods of the subject disclosure have been shown and described with reference to preferred embodiments, those skilled in the art will readily appreciate that changes and/or modifications may be made thereto without departing from the scope of the subject disclosure. For example, it is contemplated that the flexible shaft as described herein can be incorporated into tools other than fasteners, such as drills, awl drivers, or other shafted tools, and the flexible shaft need not include a head or pointed tip in such embodiments, but it may. Embodiments may include a flexible shaft alone or may be configured as a different type of driving member, such as a drill bit.

What is claimed is:

1. A flexible fastener comprising:
a shaft body having a proximal end and a distal end spaced apart along a longitudinal axis, the shaft body including a land winding helically around the shaft and having a first thickness than extends from an outer diameter of the shaft body radially inward to an inner diameter of the shaft body;
a shaft core extending from the proximal end to the distal end, the shaft core having a second thickness that extends from an outer diameter radially inward to an inner diameter, where the shaft core is arranged within the land, spaced apart from the inner diameter of the shaft body such that an annular space is defined between the inner diameter of the land and an outer diameter of the shaft core;
a flexure opening extending through the land in the radial direction through the first thickness, wherein the flexure opening extends helically about the longitudinal axis to provide for flexure of the shaft;
a torque bridge extending from the inner diameter of the shaft body radially inward to the outer diameter of the shaft core for developing toque along the shaft body when a driving torque is applied to the proximal end of the shaft body; and
a central lumen defined through the shaft core within the inner diameter of the shaft core, extending from the proximal end to the distal end.

2. The fastener as recited in claim 1, further comprising a plurality of torque bridges spaced apart helically about the longitudinal axis, wherein each torque bridge of the plurality of torque bridges are defined at a respective axial position adjacent the flexure opening.

3. The fastener as recited in claim 1, wherein the proximal end of the shaft body further includes a driving head and a shaft neck extending distally therefrom, wherein the shaft neck tapers towards a threaded portion in a distal direction, wherein the threaded portion incudes a helical thread extending radially outward from the land, and wherein the distal end further includes a narrowing tip.

4. The fastener as recited in claim 3, wherein the flexure opening is defined in the threaded portion and extends from an axial position adjacent the shaft neck helically about the longitudinal axis to an axial position adjacent the narrowing tip as a single contiguous flexure opening.

5. The fastener as recited in claim 4, wherein the flexure opening winds helically around the shaft axially offset from the external thread so that the flexure opening alternates with the external thread in an axial direction along the shaft.

6. The fastener as recited in claim 5, wherein the flexure opening extends helically in parallel with the external thread.

7. The fastener as recited in claim 3, wherein the shaft core extends from an axial position within the shaft neck, between the driving head and the threaded portion, along the longitudinal axis to the axial position adjacent the narrowing tip.

8. The fastener as recited in claim 7, further comprising a plurality of torque bridges arranged in a line spaced apart helically beginning from the axial position within the shaft neck and terminating the axial position adjacent the narrowing tip.

9. The fastener as recited in claim 1, further comprising a head at the proximal end of the shaft configured to engage a driver for turning the fastener about the longitudinal axis, wherein the distal end of the shaft defines a narrowing tip that tapers down along the longitudinal axis in a distal direction.

10. The fastener as recited in claim 19, wherein a ratio of J:I is between 3 and 4, where J is Second Polar Moment of Area (mm⁴) and I is Area Moment of Inertia (mm⁴).

11. The fastener as recited in claim 1, wherein the central lumen is dimensioned to accommodate passage of a guide wire therethrough.

12. An orthopedic implant system comprising:
an implant configured to be fastened in place with a flexible fastener, the implant including:
an implant body having opposed superior and inferior faces configured for engaging the implant between a superior vertebra and an inferior vertebra, respectively;
a bore defined through the implant body for receiving the flexible fastener, the bore extending through the implant body form a bore entrance defined in a first coronal face of the implant body and along a first axis to a bore exit defined in the superior or inferior face of the implant body and along a second axis that is angled with respect to the first axis to facilitate implantation of the implant; and
a coupling region defined in the first coronal face configured to couple an implant tool to the implant during implantation of the implant; and
an implant tool for implanting the implant, the implant tool including a driver guide for receiving a driver, and a coupling region for mating with the coupling region of the implant.

13. The orthopedic implant system as recited in claim 12, wherein the implant body includes a camming surface in the bore for turning the tip of the flexible fastener from the first axis to the second axis as the flexible fastener is advanced within the bore.

14. The orthopedic implant system as recited in claim 12, wherein the first coronal face extends between the superior and inferior faces.

15. The orthopedic implant as recited in claim 12, wherein the driver guide includes a tubular casing extending proximally from the coupling region configured to align with the bore entrance of the implant with the implant tool coupled to the implant, and
wherein the tubular casing is threaded with interior threads configured to mate with exterior threads of a driver, and
further comprising, the flexible fastener, wherein the threads of the tubular casing are timed according to threads of the flexible fastener such that the driver and flexible fastener remain engaged as they are driven.

16. The orthopedic implant system as recited in claim 15, wherein the coupling region of the implant includes a threaded female portion, and wherein the coupling region of the implant tool includes a threaded male portion configured to be threaded into the threaded female portion of the implant to couple the implant to the implant tool.

17. A method of implanting an orthopedic implant comprising:
coupling an implant to an implant tool;
inserting the implant into an orthopedic implant location, wherein the implant includes an implant body with a bore therethrough for receiving the flexible fastener, the bore extending through the implant body form a bore entrance defined in a first face of the implant body and along a first axis to a bore exit defined a second face of the implant body and along a second axis that is angled with respect to the first axis to facilitate implantation of the implant; and
affixing the implant to a bone by driving a flexible fastener in through the bore entrance along the first axis and out through the bore exit along the second axis and into the bone, wherein the flexible fastener includes a flexure opening extending helically about a longitudinal axis of the fastener to provide for flexure of the shaft.

18. The method as recited in claim 17, wherein coupling the implant to the implant tool further includes, inserting a coupling rod into a coupling region of the implant tool such that the coupling rod extends beyond the coupling region of the implant tool and into a coupling region of the implant and threading the coupling rod into the coupling region of the implant.

19. The method as recited in claim 17, wherein driving the flexible fastener includes inserting the flexible fastener and a driver into a driving guide of the implant tool and engaging the driver with a driving head of the flexible fastener within the implant to drive the flexible fastener through the implant and into bone tissue at the implant location.

20. The method as recited in claim 17, further comprising removing the coupling rod from the implant to remove the implant tool from the implant after the implant is affixed to the implant location.
